(19) United States
(12) Patent Application Publication
(10) Pub. No.: US 2019/0274782 A1
(43) Pub. Date: Sep. 12, 2019

(54) MEDICAL INSTRUMENT, MEDICAL INSTRUMENT MANAGEMENT SYSTEM, AND METHOD FOR PRODUCING THE MEDICAL INSTRUMENT

(71) Applicant: DGSHAPE Corporation, Hamamatsu-shi (JP)

(72) Inventors: Takaaki KOKUBO, Hamamatsu-shi (JP); Hisashi BITO, Hamamatsu-shi (JP); Tsutomu KUNO, Hamamatsu-shi (JP); Masaki HANAJIMA, Hamamatsu-shi (JP)

(21) Appl. No.: 16/297,968

(22) Filed: Mar. 11, 2019

(30) Foreign Application Priority Data
Mar. 12, 2018 (JP) 2018-043965

(51) Int. Cl. A61B 90/90; A61B 90/00; G16H 40/40

(52) U.S. Cl. CPC A61B 90/90; A61B 90/08; G16H 40/40; A61L 2/28; A61B 2090/0813; A61B 2090/0808; A61B 2090/0803

(57) ABSTRACT

A medical instrument includes a medical instrument main body including a resin portion made of a first resin material, and an identification mark provided on the resin portion and made of a second resin material. The medical instrument main body has predetermined information associated with the identification mark provided thereon.

### CROSS REFERENCE TO RELATED APPLICATION

This application claims the benefit of priority to Japanese Patent Application No. 2018-043965 filed on Mar. 12, 2018. The entire contents of this application are hereby incorporated herein by reference.

### BACKGROUND OF THE INVENTION

### 1. Field of the Invention

The present invention relates to a medical instrument, a medical instrument management system, and a method for producing the medical instrument.

### 2. Description of the Related Art

Conventionally, a management system is used to manage, for example, inventory of medical instruments such as instruments for surgical operation and the like, and the number of times such medical instruments have been used. Japanese Laid-Open Patent Publication No. 2005-237586, for example, discloses a management system that manages inventory of reusable instruments for surgical operation. In this management system, the reusable instruments for surgical operation are assigned identification marks, and based on the identification marks, a database is created. The number of times the instruments for surgical operation have been used is counted, so that instruments for surgical operation that have been used the number of times exceeding an upper limit of the number of times the instruments are usable are discarded or replaced.

On a medical instrument made of a metal material such as a scalpel, forceps or the like described in Japanese Laid-Open Patent Publication No. 2005-237586, an identification mark may be engraved by a laser. However, in the medical field, medical instruments made of various materials are used. For example, there are medical instruments mostly made of a resin material, medical instruments made of a metal material but including a surface not suitable for engraving, and the like. On such a medical instrument, an identification mark cannot be engraved by a laser, and thus inventory thereof cannot be managed in the manner in which the scalpel, the forceps or the like are managed.

### SUMMARY OF THE INVENTION

Preferred embodiments of the present invention provide medical instruments, inventory of which is easily managed.

The present inventors have discovered that even in a case where a medical instrument is of a type that cannot have an identification mark engraved thereon by a laser, the identification mark is allowed to be provided on a medical instrument main body including a resin portion by a different method from the engraving by the laser.

A medical instrument according to a preferred embodiment of the present invention includes a medical instrument main body including a resin portion made of a first resin material; and an identification mark provided on the resin portion and made of a second resin material, predetermined information on the medical instrument main body being associated with the identification mark.

In a medical instrument according to a preferred embodiment of the present invention, the identification mark made of the second resin material is provided on the resin portion of the medical instrument main body. The present inventors have discovered that the identification mark made of the second resin material is allowed to be provided on the resin portion made of the first resin material as described above. With such an arrangement, even in the case where the medical instrument is of a type that cannot have the identification mark engraved thereon by a laser, the identification mark made of the second resin material may be provided on the resin portion of the medical instrument main body, so that the medical instrument is managed.

The present inventors have also discovered that even in a case where the medical instrument main body is frequency cleaned in a cleaning step, namely, in a case where the identification mark is rubbed by a brush or the like or in a case where a washing detergent or the like is applied to the identification mark, the identification mark is not deteriorated at all or substantially at all. Based on this, the present inventors have discovered that the identification mark made of the second resin material may be provided on the resin portion of the medical instrument main body, so that even the medical instrument that needs to be subjected to the cleaning step is managed.

The present inventors have further discovered that the identification mark made of the second resin material is allowed to be provided on the sterilization tape used in the sterilization step. The present inventors have discovered that the identification mark is not deteriorated at all or substantially at all even after being sterilized. Based on this, the present inventors have discovered that the identification mark made of the second resin material may be provided on the sterilization tape on the medical instrument main body, so that even the medical instrument that needs to be subjected to the sterilization step is managed.

Preferred embodiments of the present invention provide medical instruments, inventory of which is easily managed.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic view showing a medical instrument management system according to a preferred embodiment of the present invention.

FIG. 2 illustrates a circulation cycle in which a medical instrument according to a preferred embodiment according to the present invention is to be used.

FIG. 3 is a schematic view showing an example of a medical instrument.

FIG. 4 is a schematic view showing another example of a medical instrument.

FIG. 5 is a schematic view showing still another example of a medical instrument.

### DETAILED DESCRIPTION OF THE PREFERRED EMBODIMENTS

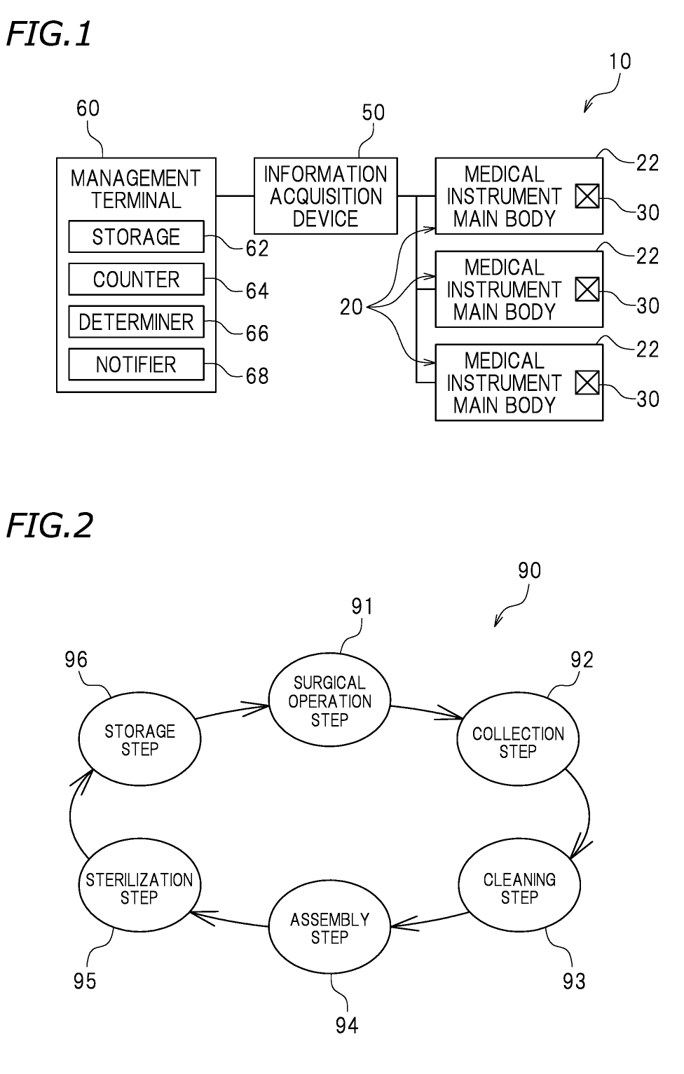

Hereinafter, preferred embodiments of medical instrument management systems according to preferred embodiments of the present invention will be described with reference to the drawings. A medical instrument management system manages information on a medical instrument (e.g., information on a medical instrument main body). As shown in FIG. 1, the medical instrument management system 10 includes a plurality of medical instruments 20, an information acquisition device 50, and a management terminal 60. The management terminal 60 and the information acquisition device 50 are communicably connected with each other in a wired manner. Alternatively, the management terminal 60 and the information acquisition device 50 may be communicably connected with each other wirelessly. The management terminal 60 manages predetermined information on a medical instrument main body 22 (described below) acquired by the information acquisition device 50. The management terminal 60 may be, for example, a notebook personal computer, a tablet terminal, a smartphone or the like.

The medical instrument main body 22 may be, for example, an instrument for surgical operation, an instrument for treatment of inpatients, a surgical instrument or the like. Examples of the instrument for surgical operation include forceps, surgical scissors, scalpels (e.g., laser scalpels and electric scalpels), ultrasonic cutters, scalpel holders, cannulae, tweezers, retractors, scales, probes, elevators, raspatories, siphons, rib spreaders, rib contractors, needle holders, injectors, metal bowls, kidney basins, cups, pins, mirrors, files, mouth gags, clamps, handpieces, elevators (elepatriums), chisels, osteotrites, raspatories, mirrors, suture needles, punch (stanze), water receiving basins, needles, (tongue) pressors, bougies, vent pipes, bone impactors, luer rongeurs, radio pliers, hammers, goniometers, fraises, droppers, metal swabs, enemators, syringes, endoscopes, and the like. Examples of the instruments for treatment of inpatients include tweezers, stethoscopes, surgical scissors, cannulae, bellows, kidney basins and the like. Examples of the surgical instrument include tweezers, stethoscopes, peans, mosquito forceps, forceps and the like. The above lists merely include examples of the medical instrument main body 22, and the medical instrument main body 22 is not limited to any of the above examples. As described above, some examples of the medical instrument main body 22, for example, tweezers, are usable as an instrument for surgical operation, as an instrument for treatment of inpatients, and as a surgical instrument.

Among various examples of the medical instrument main body 22, an instrument for surgical operation is used in a predetermined circulation cycle 90 as shown in FIG. 2. As shown in FIG. 2, the circulation cycle 90 includes a surgical operation step 91, a collection step 92, a cleaning step 93, an assembly step 94, a sterilization step 95, and a storage step 96. In the circulation cycle 90, the medical instrument main body 22 (i.e., the medical instrument 20) circulates in the order of the surgical operation step 91, the collection step 92, the cleaning step 93, the assembly step 94, the sterilization step 95, the storage step 96, and again, the surgical operation step 91. In the circulation cycle 90, typically, a medical instrument set including a plurality of the medical instruments 20 circulates. In the surgical operation step 91, a surgical operation is performed on a patient by the medical instruments 20 (i.e., the medical instrument main bodies 22) in the stored medical instrument set. In the collection step 92, the medical instruments 20 (i.e., the medical instrument main bodies 22) used for the surgical operation are collected. In the cleaning step 93, the collected medical instruments 20 (i.e., the collected medical instrument main bodies 22) are cleaned. In the assembly step 94, the cleaned medical instruments 20 (i.e., the cleaned medical instrument main bodies 22) are assembled. More specifically, some of the medical instruments 20 (i.e., some of the medical instrument bodies 22) are cleaned in a state of being divided into individual parts in the cleaning step 93, and these individual parts are assembled into the respective medical instruments 20 (i.e., the respective medical instrument main bodies 22). Also in the assembly step 94, the medical instruments 20 (i.e., the medical instrument main bodies 22) cleaned separately in the cleaning step 93 are returned so as to be positionally arranged properly in the predetermined medical instrument set. In the sterilization step 95, the medical instruments 20 (i.e., the medical instrument main bodies 22) in the above-described assembled state and/or in the above-described positionally arranged state are sterilized. In the storage step 96, the sterilized medical instruments 20 (i.e., the sterilized medical instrument main bodies 22) are stored in a predetermined storage space. In this specification, the term “assemble” is intended to encompass both of a case where the parts of a medical instrument (medical instrument main body) are assembled into one medical instrument (medical instrument main body), and a case where the plurality of medical instruments (medical instrument main bodies) are located properly in the medical instrument set as described above. The expression “assembled medical instrument” (“assembled medical instrument main body”) is intended to encompass both of a medical instrument (medical instrument main body) in a form of an assembly including the above-described parts, and a medical instrument (medical instrument main body) located properly in the medical instrument set as described above.

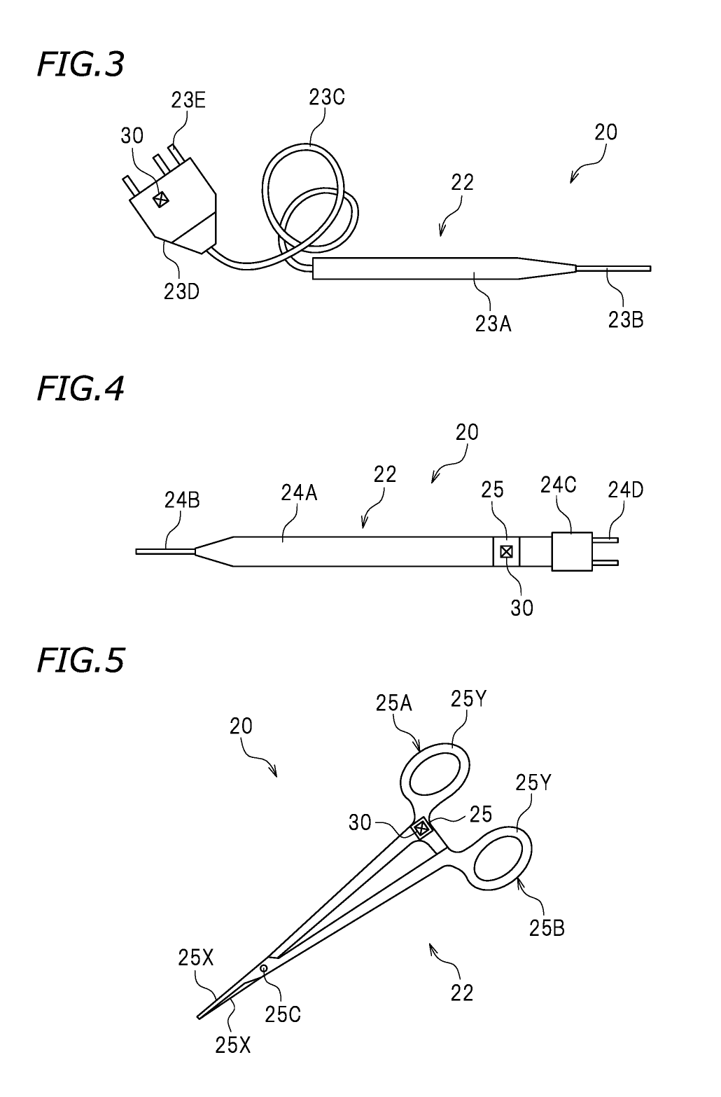

As shown in FIG. 3, the medical instrument 20 includes the medical instrument main body 22 and an identification mark 30. The medical instrument 20 shown in FIG. 3 is an electric scalpel. The medical instrument main body 22 includes a main body portion 23A, a probe 23B, a cable 23C, a connector 23D, and external connection terminals 23E. The main body portion 23A may be pencil-shaped, for example. The main body portion 23A may have an elongated cylindrical shape, for example. The main body portion 23A is to be held by a doctor. The main body portion 23A is made of a first resin material. Examples of the first resin material include polyester-based resins such as a polyethyleneterephthalate (PET) resin, a polyethylenenaphthalate (PEN) resin, and the like; polyolefin-based resins such as polyethylene (PE), polypropylene (PP), poly(vinyl chloride) (PVC), an ethylene-propylene copolymer and the like; etc. The probe 23B is provided at a tip of the main body portion 23A. The probe 23B is made of a metal material. The probe 23B is made of, for example, stainless steel, titanium or the like. The cable 23C connects the main body portion 23A and the connector 23D to each other. The cable 23C is an electric cable. The connector 23D is made of the first resin material. The resin material used to form the connector 23D and the resin material used to form the main body portion 23A may be the same as each other, or different from each other. The connector 23D is an example of a resin portion. The external connection terminals 23E are provided on the connector 23D.

The identification mark 30 is provided on the connector 23D. The identification mark 30 is made of a second resin material. The second resin material is typically different from the first resin material. The second resin material may be, for example, an acrylic resin. The second resin material has a property of not being deteriorated at all or substantially at all when, for example, being cleaned with a washing detergent or being rubbed by a brush or the like in the cleaning step 93 of the circulation cycle 90 described above. The second resin material has a property of not being deteriorated at all or substantially at all even when being sterilized in the sterilization step 95 of the circulation cycle 90. The identification mark 30 may be, for example, a two-dimensional code, a code of a GS1 coding system (e.g., numerical figure of 26 digits) or the like. The identification mark 30 is associated with predetermined information on the medical instrument main body 22. The predetermined information may be, for example, the upper limit of the number of times the medical instrument main body 22 is usable, the number of times the medical instrument main body 22 has been used, the date/time when the medical instrument main body 22 was used, the name of the operator who used the medical instrument main body 22, or the like. In the example shown in FIG. 3, the identification mark 30 is formed by foil transfer on a planar surface of the connector 23D. There is no specific limitation on the method for transferring a foil made of the second resin material (i.e., transferring the identification mark) on a member made of the first resin material. Such foil transfer may be performed by use of a conventionally known foil transfer device. In the example shown in FIG. 3, the probe 23B is a portion to contact the patient. Therefore, the identification mark 30 is provided on the connector 23D, which is not to contact the patient. The expression “contact the patient” is intended to encompass a case where the body of the patient is to contact, and a case where the blood of the patient is to contact. The expression “not to contact the patient” is intended to encompass a case where the patient is not to contact at all, and a case where the frequency of contact is much lower than the case where the patient is to contact.

As shown in FIG. 4, the medical instrument 20 includes the medical instrument main body 22 and the identification mark 30. The medical instrument 20 shown in FIG. 4 is an ultrasonic cutter, for example. The medical instrument main body 22 includes a main body portion 24A, a probe 24B, a connector 24C, and external connection terminals 24D. The main body portion 24A may be pencil-shaped, for example. The main body portion 24A may have an elongated cylindrical shape, for example. The main body portion 24A is to be held by a doctor. The main body portion 24A is made of the first resin material described above. The probe 24B is provided at a tip of the main body portion 24A. The probe 24B is made of a metal material such as stainless steel, titanium or the like. The connector 24C is made of the first resin material. The connector 24C is provided at a rear end of the main body portion 24A. The connector 24C may be cylindrical, for example. The external connection terminals 24D are provided on the connector 24C.

The main body portion 24A and the connector 24C may be cylindrical, for example. Namely, the main body portion 24A and the connector 24C include a curved surface (e.g., rounded surface) and does not include a planar surface. Therefore, it is difficult to directly foil-transfer the identification mark 30 made of the second resin material on the main body portion 24A or the connector 24C by use of a conventionally known foil transfer device. As a measure to overcome such an inconvenience, first, a sterilization tape 25 usable in the sterilization step 95 of the circulation cycle 90 described above is prepared. Then, the identification mark 30 is foil-transferred on the prepared sterilization tape 25. After this, the sterilization tape 25 having the identification mark 30 foil-transferred thereon is bonded to the curved surface of the main body portion 24A. As can be seen, even if the medical instrument main body 22 has a shape with no planar surface, the sterilization tape 25 may be used to provide the identification mark 30 on the medical instrument main body 22 by foil transfer. In the example shown in FIG. 4, the probe 24B is a portion to contact the patient. Therefore, the identification mark 30 is provided at the rear end, of the main body portion 24A, which is not to contact the patient.

The sterilization tape 25 has a color of at least a portion thereof changed when being sterilized in the sterilization step 95. The sterilization tape 25 has a property of not being deteriorated at all or substantially at all by being sterilized. Methods for sterilization include, for example, gas sterilization with ethylene oxide gas, plasma sterilization, thermal sterilization by use of an autoclave, and the like. The sterilization tape 25 includes an adhesive layer to adhere to another member and a resin layer made of the first resin material. The sterilization tape 25 is an example of a resin portion. The sterilization tape 25 may be, with no specific limitation, any sterilization tape usable in a sterilization step in the medical field, as long as including a resin layer made of the first resin material.

As shown in FIG. 5, the medical instrument 20 includes the medical instrument main body 22 and the identification mark 30. The medical instrument 20 shown in FIG. 5 is forceps, for example. The medical instrument main body 22 includes a pair of forceps portions 25A and 25B and a pin 25C. The pair of forceps portions 25A and 25B are made of a metal material such as stainless steel, titanium or the like. The pair of forceps portions 25A and 25B are pivotably supported by the pin 25C.

The pair of forceps portions 25A and 25B are made of a metal material although including a planar surface. Therefore, it is difficult to directly foil-transfer the identification mark 30 made of the second resin material on the forceps portion 25A or 25B by use of a conventionally known foil transfer device. As a measure to overcome such an inconvenience, first, the sterilization tape 25 is prepared. Then, the identification mark 30 is foil-transferred on the sterilization tape 25. After this, the sterilization tape 25 having the identification mark 30 foil-transferred thereon is bonded to the planar surface of, for example, the forceps portion 25A. As can be seen, even if the medical instrument main body 22 is made of a metal material, the sterilization tape 25 may be used to provide the identification mark 30 on the medical instrument main body 22 by foil transfer. In the example shown in FIG. 5, the sterilization tape 25 is bonded to the forceps portion 25A after the identification mark 30 is foil-transferred on the sterilization tape 25. The present invention is not limited to this. For example, the identification mark 30 may be foil-transferred on the sterilization tape 25 after the sterilization tape 25 is bonded to the planar surface of the forceps portion 25A. In the example shown in FIG. 5, blades 25X of the pair of forceps portions 25A and 25B are portions to contact the patient. Therefore, the identification mark 30 is provided on one of handles 25Y, which are not to contact the patient. The handles 25Y are to be held by the doctor and are provided at ends, of the pair of forceps portions 25A and 25B, opposite to the blades 25X.

The information acquisition device 50 acquires the above-described predetermined information on the medical instrument main body 22 from the identification mark 30 of the medical instrument 20. The information acquisition device 50 transmits the predetermined information on the medical instrument main body 22, acquired from the identification mark 30, to the management terminal 60. The information acquisition device 50 may be any device with no specific limitation as long as being capable of acquiring predetermined information on the medical instrument main body 22 from the identification mark 30. In a case where, for example, the identification mark 30 is a two-dimensional code, the information acquisition device 50 may be a two-dimensional code reader capable of reading a two-dimensional code. The act of acquiring the predetermined information on the medical instrument main body 22 from the identification mark 30 by use of the information acquisition device 50 is performed in, for example, at least one step of the circulation cycle 90 described above.

As shown in FIG. 1, the management terminal 60 includes a storage 62, a counter 64, a determiner 66 and a notifier 68. These elements may be provided in the form of software. Namely, these elements may be realized by the management terminal 60 (computer) as a result of a computer program or programs being read into the management terminal 60. The computer program or programs may be stored on a computer-readable storage medium such as a CD, a DVD or the like. The computer program may be downloadable via the Internet. Alternatively, the above-described elements may be realized by a processor and/or a circuit included in the management terminal 60.

The storage 62 stores the predetermined information on each of the medical instrument main bodies 22 acquired by the information acquisition device 50. The storage 62 stores the number of times each medical instrument main body 22 has been used.

The counter 64 counts the number of times each medical instrument main body 22 has been used.

The determiner 66 determines whether or not the number of times each medical instrument main body 22 has been used has reached a predetermined upper limit.

In a case where the determiner 66 determines that the number of times the medical instrument main body 22 has been used has reached the predetermined upper limit, the notifier 68 notifies the operator that the medical instrument main body 22 is to be replaced or discarded. There is no specific limitation on the method for notification. The method for notification may be, for example, visual display, voice or the like. In this preferred embodiment, the operator is visually notified by a display device (not shown).

As described above, the medical instrument 20 according to this preferred embodiment includes the identification mark 30 made of the second resin material provided on the connector 23D of the medical instrument main body 22. The present inventors have discovered that the identification mark 30 made of the second resin material is allowed to be provided on the connector 23D made of the first resin material as described above. With such an arrangement, even in the case where the medical instrument 20 is of a type that cannot have the identification mark 30 engraved thereon by a laser, the identification mark 30 made of the second resin material is allowed to be provided on the connector 23D of the medical instrument main body 22, so that the medical instrument 20 is managed.

The medical instrument 20 according to this preferred embodiment is used in the predetermined circulation cycle 90 including the cleaning step 93 of cleaning the medical instrument main body 22. The present inventors have discovered that even if the medical instrument main 22 is cleaned in the cleaning step 93, the identification mark 30 is not deteriorated at all or substantially at all, and has confirmed that the medical instrument is managed with no problem by the identification mark 30 provided on the resin portion of the medical instrument main body 22, which needs to be subjected to the cleaning step 93.

In the medical instrument 20 according to this preferred embodiment, the medical instrument main body 22 includes the sterilization tape 25, and the identification mark 30 made of the second resin material is provided on the sterilization tape 25. The present inventors have discovered that the identification mark 30, even in the case of being provided on the sterilization tape 25, is not deteriorated at all or substantially at all by the cleaning step 93 or the sterilization step 95, and has confirmed that the medical instrument 20 is managed with no problem by the identification mark 30 provided on the sterilization tape 25 of the medical instrument main body 22, which needs to be subjected to the cleaning step 93 and the sterilization step 95.

The medical instrument 20 according to this preferred embodiment is used in the predetermined circulation cycle 90 including the surgical operation step 91, the collection step 92, the cleaning step 93, the assembly step 94, the sterilization step 95, and the storage step 96. In the steps 91 through 96, the management of the medical instrument 20 is especially important. The medical instrument 20 is easily managed by the identification mark 30, so that the load on the operator is alleviated.

In the medical instrument 20 according to this preferred embodiment, the medical instrument main body 22 includes a curved surface on which the sterilization tape 25 is provided. Even in the case where the medical instrument main body 22 is made of a metal material, engraving cannot be made on a curved surface by a laser usually. However, the identification mark 30 may be provided on the sterilization tape 25 and the sterilization tape 25 may be, for example, bonded to the curved surface, so that the identification mark 30 is provided on the curved surface.

In the medical instrument 20 according to this preferred embodiment, the medical instrument main body 22 includes the probe 23B to contact the patient and the connector 23D not to contact the patient. The identification mark 30 is provided on the connector 23D. Since the identification mark 30 is provided on the connector 23D, which is not to contact the patient, the identification mark 30 is more reliably prevented from being deteriorated than in the case of being provided on the probe 23B.

In the medical instrument 20 according to this preferred embodiment, the second resin material used to form the identification mark 30 preferably is an acrylic resin, for example. The present inventors have discovered that the identification mark 30, in the case of being made of an acrylic resin, is usable for a long time.

In the medical instrument 20 according to this preferred embodiment, the predetermined information associated with the identification mark 30 includes the upper limit of the number of times the medical instrument main body 22 is usable. With such an arrangement, the time to replace or discard the medical instrument 20 is correctly recognizable.

Preferred embodiments of the present invention are described above. The above-described preferred embodiments are merely examples, and the present invention may be carried out in any of various other preferred embodiments.

In the above-described preferred embodiments, the identification mark 30 is provided in a portion that is not to contact the patient. In the case where, for example, the medical instrument main body 22 is an assembly of two or more members, the identification mark 30 is not provided on a portion at which such members are joined with each other, or on a portion not visually recognizable from outside, among the portions not to contact the patient.

The terms and expressions used herein are for description only and are not to be interpreted in a limited sense. These terms and expressions should be recognized as not excluding any equivalents to the elements shown and described herein and as allowing any modification encompassed in the scope of the claims. The present invention may be embodied in many various forms. This disclosure should be regarded as providing preferred embodiments of the principles of the present invention. These preferred embodiments are provided with the understanding that they are not intended to limit the present invention to the preferred embodiments described in the specification and/or shown in the drawings. The present invention is not limited to the preferred embodiments described herein. The present invention encompasses any of preferred embodiments including equivalent elements, modifications, deletions, combinations, improvements and/or alterations which can be recognized by a person of ordinary skill in the art based on the disclosure. The elements of each claim should be interpreted broadly based on the terms used in the claim, and should not be limited to any of the preferred embodiments described in this specification or used during the prosecution of the present application.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A medical instrument, comprising:
a medical instrument main body including a resin portion made of a first resin material; and
an identification mark provided on the resin portion and made of a second resin material; wherein
predetermined information is provided on the medical instrument main body and is associated with the identification mark.

2. The medical instrument according to claim 1, wherein the medical instrument is used in a predetermined circulation cycle including a cleaning step of cleaning the medical instrument main body.

3. The medical instrument according to claim 2, wherein
the predetermined circulation cycle further includes a sterilization step of sterilizing the medical instrument main body;
the sterilization step uses a sterilization tape with a color of at least a portion thereof being changed when being sterilized; and
the resin portion is the sterilization tape.

4. The medical instrument according to claim 3, wherein the predetermined circulation cycle further includes:
a surgical operation step of performing a surgical operation by use of the medical instrument main body;
a collection step of collecting the medical instrument main body used for the surgical operation;
an assembly step of assembling the cleaned medical instrument main body; and
a storage step of storing the sterilized medical instrument main body; wherein
the cleaning step cleans the collected medical instrument main body; and
the sterilization step sterilizes the assembled medical instrument main body.

5. The medical instrument according to claim 3, wherein the medical instrument main body includes a curved surface on which the sterilization tape is provided.

6. The medical instrument according to claim 1, wherein
the medical instrument main body includes a first portion to contact a patient and a second portion not to contact the patient; and
the identification mark is provided on the second portion.

7. The medical instrument according to claim 1, wherein the second resin material is an acrylic resin.

8. The medical instrument according to claim 1, wherein the predetermined information includes an upper limit of a number of times the medical instrument main body is usable.

9. A medical instrument management system, comprising:
the medical instrument according to claim 1;
an information acquisition device that acquires the predetermined information from the identification mark; and
a management terminal that manages the predetermined information acquired by the information acquisition device.

10. A method for producing a medical instrument comprising:
preparing a medical instrument main body including a resin portion made of a first resin material; and
foil-transferring, on the resin portion, an identification mark made of a second resin material; wherein
predetermined information is formed on the medical instrument main body and is associated with the identification mark.

11. The method for producing a medical instrument according to claim 10, wherein the resin portion is a sterilization tape with a color of at least a portion thereof being changed when being sterilized.

12. The method for producing a medical instrument according to claim 10, wherein the medical instrument main body is used in a predetermined circulation cycle including a surgical operation step of performing a surgical operation by use of the medical instrument main body, a collection step of collecting the medical instrument main body used for the surgical operation, a cleaning step of cleaning the collected medical instrument main body, an assembly step of assembling the cleaned medical instrument main body, a sterilization step of sterilizing the assembled medical instrument main body, and a storage step of storing the sterilized medical instrument main body.

13. A method for producing a medical instrument comprising:
preparing a medical instrument main body;
preparing a sterilization tape with a color of at least a portion thereof being changed when being sterilized;
foil-transferring an identification mark made of a resin material on the sterilization tape, predetermined information formed on the medical instrument main body being associated with the identification mark; and
bonding the sterilization tape including the identification mark foil-transferred thereon to the medical instrument main body.

14. The method for producing a medical instrument according to claim 13, wherein the medical instrument main body is used in a predetermined circulation cycle including a surgical operation step of performing a surgical operation by use of the medical instrument main body, a collection step of collecting the medical instrument main body used for the surgical operation, a cleaning step of cleaning the collected medical instrument main body, an assembly step of assembling the cleaned medical instrument main body, a sterilization step of sterilizing the assembled medical instrument main body, and a storage step of storing the sterilized medical instrument main body.
